(19) United States
(12) Patent Application Publication
(10) Pub. No.: US 2016/0326085 A1
(43) Pub. Date: Nov. 10, 2016

(54) 2-ARYL-ZINC-PROPIONATE CATALYST AND PREPARATION METHOD AND USE THEREOF

(71) Applicant: FUDAN UNIVERSITY, Shanghai (CN)

(72) Inventors: Fener CHEN, Shanghai (CN); Qiuqin HE, Shanghai (CN); Fangjun XIONG, Shanghai (CN); Wenxue CHEN, Shanghai (CN)

(21) Appl. No.: 15/106,194

(22) PCT Filed: Dec. 18, 2013
(86) PCT No.: PCT/CN2013/089773

§ 371 Date: Jun. 17, 2016

(51) Int. Cl. C07C 57/30; B01J 37/04; C07C 57/40; C07C 57/58; C07C 59/64; C07C 59/86; C07C 59/68; B01J 31/04; C07C 51/41

(52) U.S. Cl. CPC C07C 57/30; B01J 31/04; B01J 37/04; C07C 57/40; C07C 51/418; C07C 51/412; C07C 59/64; C07C 59/86; C07C 59/68; C07C 57/58; B01J 2231/40; B01J 2531/004; C07C 2101/08

(57) ABSTRACT

The present invention belongs to the technical field of chemical catalysts, and particularly relates to a zinc 2-arylpropionate catalyst, a preparation method therefor and use thereof The structural formula of the zinc 2-arylpropionate catalyst of the present invention is one of the following structures. The catalyst can be used for homogeneous catalysis of a 1,2-aryl rearrangement reaction of α-haloarylketal, and especially for synthesis of high yield and environmentally friendly 2-arylpropanonic acid non-steroidal anti-inflammatory analgesic drugs, such as, ibuprofen, ketoprofen, loxoprofen, flurbiprofen, fenoprofen, or naproxen and the like.

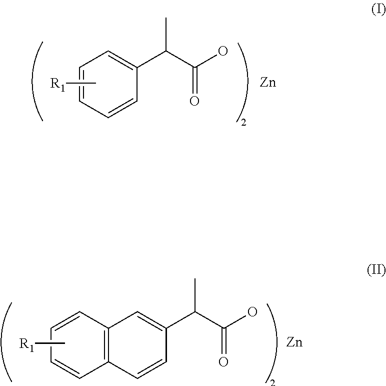

### FIELD OF THE INVENTION

The present invention belongs to the technical field of chemical catalysts, and particularly relates to a zinc 2-arylpropionate catalyst, a preparation method therefor and use thereof, especially the use thereof in the syntheses of 2-arylpropanonic acid non-steroidal anti-inflammatory analgesic drugs.

### BACKGROUND ARTS

2-Arylpropanonic acid non-steroidal anti-inflammatory analgesic drugs, such as ibuprofen, ketoprofen, loxoprofen, flurbiprofen, fenoprofen, naproxen, etc., are clinically widely used for treating arthritis, rheumatoid arthritis, and the relief of various fever and pain symptoms.

In the presence of a catalyst, 1,2-aryl rearrangement of an α-haloarylketal is a classical method for preparing a 2-arylpropanonic acid non-steroidal anti-inflammatory analgesic drug. Giordano used AgBF₄ to catalyze the 1,2-aryl rearrangement of an α-haloarylketal, but AgBF₄ has a too expensive price, and is not suitable for industrial productions (J. Chem. Soc. Perkin Trans. 1, 1982, 11, 2575; Tetrahedron, 1982, 23, 1385). There are documents reporting that lewis acids, such as anhydrous ZnCl₂, ZnBr₂ or SnCl₄, can catalyze the 1,2-aryl rearrangement of an α-haloarylketal, but there are colloidal black insolubles produced during the reaction, and the separating operation is relatively complex (European patent EP 0035305, 0034871, and 0163338; U.S. Pat. No. 4,414,405; J. Org. Chem. 1983, 48, 4658; and Chinese Journal of Pharmaceuticals, 1988, 19, 483). CHEN, Fener et al. (Chinese patent ZL 00127293.4; Chinese Journal of Pharmaceuticals, 1998, 29, 531; West China Journal of Pharmaceutcal Sciences, 1995, 10, 129); and Piccolo (J. Org. Chem., 1987, 52, 10) and European patent 0174844 describe the use of ZnO, ZnS or Zn(OH)₂ to catalyze such a rearrangement reaction, with a higher rearrangement yield, but due to the presence of a heterogeneous catalyst, the reaction needs a higher temperature, material outshoot easily occurs, and the product quality is relatively poor. US patent U.S. Pat. No. 4,623,736 discloses the rearrangement of an α-chlorinated isobutylphenyl phenyl ketal under the catalysis of zinc 2-ethylhexanoate to prepare ibuprofen, but this reaction has strict conditions and is carried out almost without a solvent, and the product is a black oily substance and has a poorer quality. Zinc tosylate or a mixture of same and ZnO is used in Chinese patent ZL 92106667.8 to catalyze the molecular rearrangement so as to prepare ibuprofen, which needs to be performed at a high temperature of 140-150° C.

### SUMMARY OF THE INVENTION

An object of the present invention lies in overcoming the drawbacks in the prior art, and providing a homogeneous catalyst of zinc 2-arylpropionate, a preparation therefor and use thereof, especially the use thereof in the syntheses of 2-arylpropanonic acid non-steroidal anti-inflammatory analgesic drugs.

Such a zinc 2-arylpropionate catalyst provided by the present invention has one of the following structures:

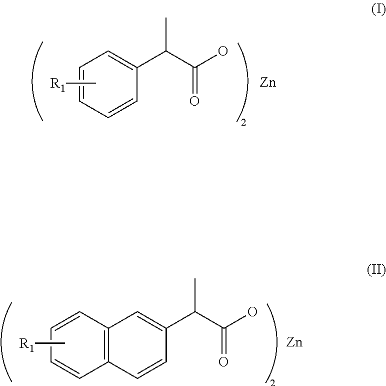

wherein R₁ and R₂ are respectively selected from hydrogen, a C₁-C₄ alkyl, a methoxyl, a trifluoromethyl, a halogen, a phenyl, and a benzyl; R₁ is ortho-substituted, meta-substituted or para-substituted, mono-substituted or polysubstituted; and R₂ is C-5, C-6, C-7, or C-8 substituted, mono-substituted or polysubstituted.

The synthetic route of such a zinc 2-arylpropionate catalyst of the present invention is as follows:

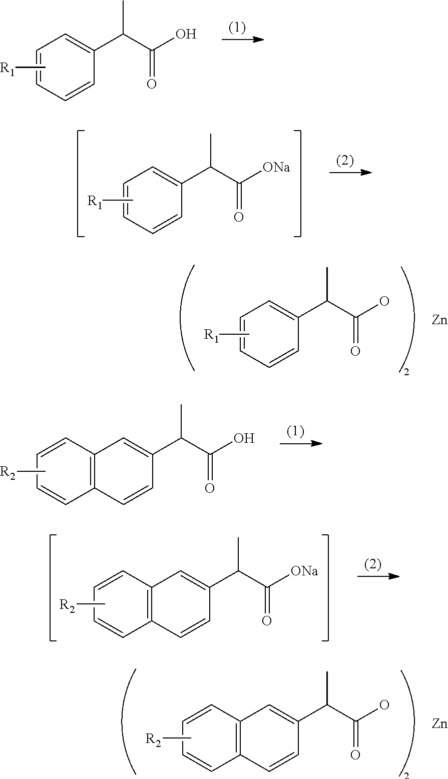

the specific steps are:

(1) at room temperature, an alkaline earth hydroxide is dissolved in a C₁-C₄ alcohol, 2-arylpropionic acid is added, and reacted at room temperature to 100° C. for 0.5-2 h to obtain a solution of alkaline earth 2-arylpropionate. Said alkaline earth hydroxide is sodium hydroxide, potassium hydroxide or calcium hydroxide, and the molar ratio of the 2-arylpropionic acid to the alkaline earth hydroxide is 1:0.8-1:1.5.

(2) a zinc salt or zinc oxide is added into the above-mentioned alkaline earth 2-arylpropionate solution, and reacted at room temperature to 100° C. for 0.5-20 h to obtain zinc 2-arylpropionate; said zinc salt is zinc chloride, zinc sulphate or zinc acetate; and the molar ratio of said zinc salt or zinc oxide to the alkaline earth 2-arylpropionate is 0.5:1-0.5:1.2.

Preferred reaction conditions of the present invention are: in step (1), the alkaline earth hydroxide is sodium hydroxide, the C₁-C₄ alcohol is methanol or ethanol, the reaction temperature is 45-55° C., the reaction time is 0.5-2 h, and the molar ratio of the 2-arylpropionic acid to the alkaline earth hydroxide is 1:1-1:1.1. In step (2), said zinc salt is zinc acetate, and the molar ratio of the zinc acetate to the sodium 2-arylpropionate is 0.5:1-0.5:1.1.

The zinc 2-arylpropionate catalyst of the present invention can be used for catalyzing the 1,2-aryl rearrangement reaction of the corresponding α-haloarylketal (III). Especially, by said rearrangement reaction, 2-arylpropanonic acid non-steroidal anti-inflammatory analgesic drugs are synthesized by catalysis. Particularly, the zinc 2-arylpropionate and α-haloarylketal are reacted in a single or mixed aromatic hydrocarbon solvent at 80-160° C. for 1-10 h. Said aromatic hydrocarbon solvent is benzene, mono-substituted benzene or multisubstituted benzene, etc. The molar ratio of said zinc 2-arylpropionate catalyst to the α-haloarylketal is 0.01:1-0.5:1.

In the present invention, preferred reaction conditions of the rearrangement reaction are: the aromatic hydrocarbon solvent is toluene, the reaction temperature is 110-120° C., and the molar ratio of the catalyst to the α-haloarylketal is 0.02:1-0.1:1. The α-haloarylketal can be prepared with reference to methods in the following documents: U.S. Pat. No. 4,623,736; J. Chem. Soc. Perkin Trans. 1, 1986, 1983; CHEN, Fener et al., Chinese Journal of Pharmaceuticals, 1996, 27, 195; Chinese Journal of Pharmaceuticals, 1998, 29, 531; West China Journal of Pharmaceutcal Sciences, 1995, 10, 129; and XIONG Xianqiang et al., Chinese Journal of Pharmaceuticals, 2000, 31, 436. The α-haloarylketal can be selected from one of the following structures:

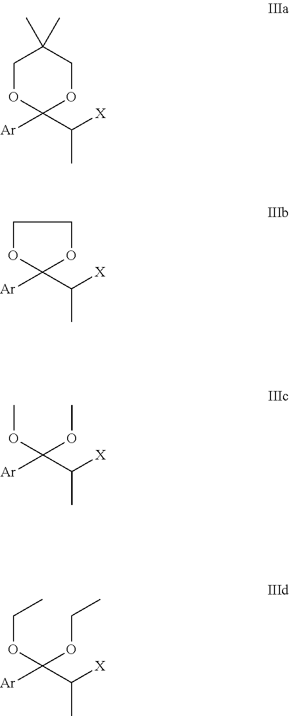

wherein Ar is

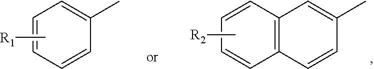

and X is Cl, Br or I. The zinc 2-arylpropionate catalyst provided by the present invention is simple to be synthesized, and can be used for synthesis of 2-arylpropanonic acid non-steroidal anti-inflammatory analgesic drugs by homogeneous catalysis in a high yield under moderate conditions.

### DETAILED DESCRIPTION OF THE INVENTION

The present invention is further described below in detail in combination with particular embodiments, but the scope of protection of the present invention is not limited thereto.

### Embodiment 1 Synthesis of zinc 2-phenylpropionate

0.4 g (0.01 mol) of sodium hydroxide and 15 mL of methanol are added into a 100 mL round-bottom flask, and dissolved at room temperature under stirring, thereafter 1.5 g (0.01 mol) of 2-phenylpropionic acid is added and reacted at 50° C. for 0.5 h, and cooled to room temperature. 1.1 g (0.005 mol) of a solution of zinc acetate dihydrate dissolved in methanol (10 mL) is dropwise added and reacted at 50° C. for 1 h after the completion of the addition, the majority of the solvent is recovered, and a white solid is filtered out, washed with a methanol solution, and dried at 150° C. under a reduced pressure for 7 h to obtain 1.81 g of zinc 2-phenylpropionate with a yield of 99.5% and m.p. 262-264° C.

### Embodiment 2 Synthesis of zinc 2-p-tolylpropionate

0.44 g (0.011 mol) of sodium hydroxide and 15 mL of methanol are added into a 100 mL round-bottom flask, and dissolved at room temperature under stirring, thereafter 1.64 g (0.01 mol) of 2-p-tolylpropionic acid is added and reacted at 50° C. for 2 h, and cooled to room temperature. 1.1 g (0.005 mol) of a solution of zinc acetate dihydrate dissolved in methanol is dropwise added (10 mL) and undergoes a reflux reaction for 7 h after the completion of the addition, the majority of the solvent is recovered, and a white solid is filtered out, washed with a methanol solution, and dried at 150° C. under a reduced pressure for 7 h to obtain 1.66 g of zinc 2-p-tolylpropionate with a yield of 84.7% and m.p. 256-258° C.

### Embodiment 3 Synthesis of zinc 2-p-isobutylphenylpropionate

44 mg (1.1 mmol) of sodium hydroxide and 8 mL of methanol are added into a 25 mL round-bottom flask, and dissolved at room temperature under stirring, thereafter 206 mg (1 mmol) of 2-p-isobutylphenylpropionic acid is added and reacted at 50° C. for 2 h, and cooled to room temperature. 110 mg (0.5 mmol) of a solution of zinc acetate dihydrate dissolved in methanol is dropwise added (2 mL) and undergoes a reflux reaction for 10 h after the completion of the addition, the majority of the solvent is recovered, and a white solid is filtered out, washed with a methanol solution, and dried at 150° C. under a reduced pressure for 7 h to obtain 192 mg of zinc 2-p-isobutylphenylpropionate with a yield of 80.8% and m.p. 203-206° C.

### Embodiment 4 Synthesis of zinc 2-(6-methoxylnaphthyl)propionate

40 mg (1 mmol) of sodium hydroxide and 8 mL of methanol are added into a 25 mL round-bottom flask, and dissolved at room temperature under stirring, thereafter 230 mg (1 mmol) of 2-(6-methoxylnaphth-2-yl)propionic acid is added and reacted at 50° C. for 2 h, and cooling is performed to room temperature. 110 mg (0.5 mmol) of a solution of zinc acetate dihydrate dissolved in methanol is dropwise added (2 mL) and undergoes a reflux reaction for 7 h after the completion of the addition, the majority of the solvent is recovered, and a white solid is filtered out, washed with a methanol solution, and dried at 150° C. under a reduced pressure for 7 h to obtain 183 mg of zinc 2-(6-methoxylnaphth-2-yl)propionate with a yield of 69.8% and m.p. 205-207° C.

Examples of the use of zinc 2-arylpropionate in the catalysis of 1,2-aryl rearrangement reaction of the corresponding α-haloarylketal are as follows.

### Embodiment 5 Synthesis of 2-(6-methoxylnaphth-2-yl)naphthylpropionic acid ((±)naproxen) under catalysis of zinc 2-(6-methox-2-yl)naphthyl propionate

16.7 g (0.05 mol) of 2-(1 -bromoethyl)-2-(6-methoxylnaphth-2-yl)-5,5-dimethyl-1,3 -epoxyhexane, 0.523 g (0.001 mol) of zinc 2-(6-methoxylnaphthyl)propionate and 50 mL of toluene are added into a 500 mL round-bottom flask and undergo a reflux reaction for 5.5 h, a 30% sodium hydroxide solution (50 mL) is added, after refluxing under stirring for 3.5 h and cooling to 50° C., water (30 mL) and a small amount of activated carbon are added, and the stirring refluxing is continued for 0.5 h. After cooling, filtration and standing, an aqueous layer is separated, a toluene layer is washed with water (50 mL×3), the aqueous layers are combined and adjusted with concentrated hydrochloric acid to a pH of 1-2 to separate a white solid, and the white solid is filtered, the crude product is washed with water and dried. Recrystallization is performed using ethanol-water to obtain 11.14 g of a white powder with a yield of 96.9%. The m.p. is 152-154° C.

### Embodiment 6 Synthesis of 2-(4-methoxylphenyl)propionic acid (critical intermediate of loxoprofen) under catalysis of zinc 2-(4-methylphenyl)propionate

13.4 g (0.05 mol) of 2-(1-chloroethyl)-5,5-dimethyl-2-p-tolyl-1,3-epoxyhexane, 0.523 g (0.001 mol) of zinc 2-(6-methoxylnaphthyl)propionate and 50 mL of toluene are added into a 500 mL round-bottom flask and undergo reflux reaction for 3.5 h, a 30% sodium hydroxide solution (50 mL) is added, after reflux under stirring for 3.5 h and cooling to 50° C., water (30 mL) and a small amount of activated carbon are added, and agitation reflux continues for 0.5 h. After cooling, filtration and standing, aqueous layer is separated, a toluene layer is washed with water (50 mL×3), the aqueous layers are combined and adjusted with a concentrated hydrochloric acid to a pH of 1-2 extracted with chloroform (30 mL×3), dried using an anhydrous sodium sulphate, the chloroform is distilled off to obtain 7.92 g of a white solid with a yield of 96.6%. The m.p. is 37-38° C.

### Embodiment 7 Synthesis of 2-(4-isobutylphenyl)propionic acid (ibuprofen) under catalysis of zinc 2-(4-isobutylphenyl)propionate

15.6 g (0.05 mol) of 2-(1-bromoethyl)-2-p-butylphenyl-1,3-epoxypentane, 0.784 g (0.0015 mol) of zinc 2-(6-methoxylnaphthyl)propionate and 50 mL of toluene are added into a 500 mL round-bottom flask and undergo a reflux reaction for 3.0 h, a 30% sodium hydroxide solution (50 mL) is added, after refluxing under stirring for 3.5 h and cooling to 50° C., water (30 mL) and a small amount of activated carbon are added, and the stirring refluxing is continued for 0.5 h. After cooling, filtration and standing, an aqueous layer is separated, a toluene layer is washed with water (50 mL×3), the aqueous layers are combined and adjusted with concentrated hydrochloric acid to a pH of 1-2 to separate a white solid, and the white solid is filtered, the crude product is washed with water and dried. Recrystallization is performed using ethanol-water to obtain 10.01 g of a white powder with a yield of 97.2%. The m.p. is 74-75° C.

### Embodiment 8 Synthesis of 2-(3-phenoxyl)propionic acid (fenoprofen) under catalysis of zinc 2-(3-phenoxyl)propionate

8.52 g (0.02 mol) of 1-(1,1-diethyoxyl-2-iodopropyl)-3-phenoxybenzene, 0.219 g (0.0004 mol) of zinc 2-(6-methoxylnaphthyl)propionate and 30 mL of toluene are added into a 500 mL round-bottom flask and undergo a reflux reaction for 3.5 h, a 30% sodium hydroxide solution (50 mL) is added, after refluxing under stirring for 3.5 h and cooling to 50° C., water (15 mL) and a small amount of activated carbon are added, and the stirring refluxing is continued for 0.5 h. After cooling, filtration and standing, an aqueous layer is separated, a toluene layer is washed with water (30 mL×3), the aqueous layers are combined and adjusted with concentrated hydrochloric acid to a pH of 1-2 extracted with chloroform (30 mL×3), dried using an anhydrous sodium sulphate, the chloroform is distilled off to obtain 4.48 g of a light yellow liquid with a yield of 92.5%. ¹H NMR (CDCl₃) δ 1.49 (d, 3H, CH₃), 3.71 (q, 1H, CH), 6.87-7.37 (m, 9H, ArH), 7.50-8.40 (brs, COOH).

### Embodiment 9 Synthesis of 2-(3-fluoro-4-phenyl)phenylpropionic acid (flurbiprofen) under catalysis of zinc 2-(3-fluoro-4-phenyl)propionate

7.84 g (0.02 mol) of 2-(1 -bromoethyl)-2-(2-fluoro-(1,1′-biphen-4-yl)-5,5-dimethyl-1,3-dioxocyclohexan e, 0.276 g (0.0005 mol) of zinc 2-(6-methoxylnaphthyl)propionate and 30 mL of toluene are added into a 500 mL round-bottom flask and undergo a reflux reaction for 3.5 h, a 30% sodium hydroxide solution (50 mL) is added, after refluxing under stirring for 3.5 h and cooling to 50° C., water (15 mL) and a small amount of activated carbon are added, and the stirring refluxing is continued for 0.5 h. After cooling, filtration and standing, an aqueous layer is separated, a toluene layer is washed with water (30 mL×3), the aqueous layers are combined and adjusted with concentrated hydrochloric acid to a pH of 1-2 to separate a white solid, and the white solid is filtered, the crude product is washed with water and dried. Recrystallization is performed using ethanol-water to obtain 4.45 g of a white powder with a yield of 91.2%. The m.p. is 110-112° C.

1. A zinc 2-arylpropionate catalyst of formula (I) or (II) 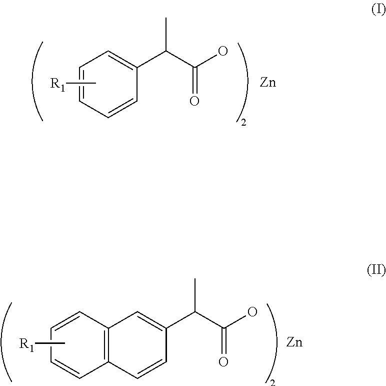
wherein R₁ and R₂ are respectively selected from hydrogen, a C₁-C₄ alkyl, a methoxyl, a trifluoromethyl, a halogen, a phenyl, a benzyl; R₁ is ortho-substituted, meta-substituted or para-substituted, mono-substituted or polysubstituted; and R₂ is C-5, C-6, C-7, or C-8 substituted, mono-substituted or polysubstituted.

2. A method for preparing the zinc 2-arylpropionate catalyst in accordance with claim 1, comprising the steps of:
(1) an alkaline earth hydroxide is dissolved in a C₁-C₄ alcohol, 2-arylpropionic acid is added, the reaction being for 0.5-2 h at room temperature to 100° C. to obtain a solution of alkaline earth 2-arylpropionate; said alkaline earth hydroxide is lithium hydroxide, sodium hydroxide, potassium hydroxide or calcium hydroxide, and the molar ratio of the 2-arylpropionic acid to the alkaline earth hydroxide is 1:0.8-1:1.5;
(2) a zinc salt or zinc oxide is added into the above-mentioned alkaline earth 2-arylpropionate solution, the reaction being for 0.5-20 h at room temperature to 100° C. to obtain zinc 2-arylpropionate; said zinc salt is zinc chloride, zinc sulphate or zinc acetate; and the molar ratio of said zinc salt or zinc oxide to the alkaline earth 2-arylpropionate is 0.5:1-0.5:1.2.

3. The method of claim 2, characterized in that in step (1), the alkaline earth hydroxide is sodium hydroxide, the C₁-C₄ alcohol is methanol or ethanol, the reaction temperature is 45-55° C., the reaction time is 0.5-2 h, and the molar ratio of the 2-arylpropionic acid to the alkaline earth hydroxide is 1:1-1:1.1.

4. The method of claim 2 or 3, characterized in that in step (2), said zinc salt is zinc acetate, and the molar ratio of the zinc acetate to the sodium 2-arylpropionate is 0.5:1-0.5:1.1.

5. Use of the zinc 2-arylpropionate catalyst of claim 1 in homogeneous catalysis of a 1,2-aryl rearrangement reaction of an α-haloarylketal, characterized in that zinc 2-arylpropionate and an α-haloarylketal are reacted for 1-10 h in a single or mixed aromatic hydrocarbon solvent at 80-160° C. to obtain a 2-arylpropanonic acid non-steroidal anti-inflammatory analgesic drug; said aromatic hydrocarbon solvent is benzene, mono-substituted benzene or multisubstituted benzene; and the molar ratio of the zinc 2-arylpropionate catalyst to the α-haloarylketal is 0.01:1-0.5:1.

6. The use of claim 5, characterized in that said aromatic hydrocarbon solvent is toluene, the reaction temperature is 110-120° C., and the molar ratio of the catalyst to the α-haloarylketal is 0.02:1-0.1:1.

7. The use of claim 5 or 6, characterized in that said α-haloarylketal has one of the following structures: 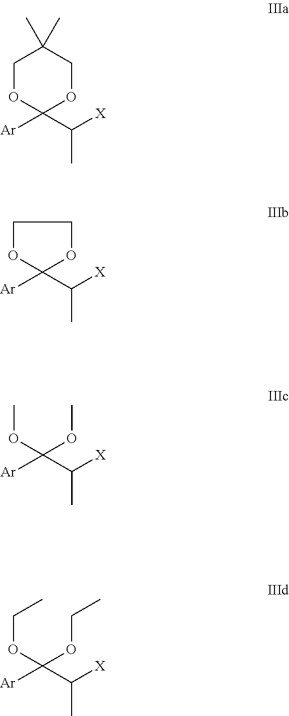
wherein Ar is 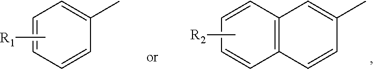
and X is Cl, Br or I.

8. The use of claim 5 or 6, characterized in that said 2-arylpropanonic acid non-steroidal anti-inflammatory analgesic drug is ibuprofen, ketoprofen, loxoprofen, flurbiprofen, fenoprofen, or naproxen.
